(19) United States
(12) Patent Application Publication
(10) Pub. No.: US 2018/0251808 A1
(43) Pub. Date: Sep. 6, 2018

(54) Method and Reagents for Detecting Contaminants in Oil and Gas Environments

(71) Applicant: Dow Global Technologies LLC, Midland, MI (US)

(72) Inventors: Geert Van Der Kraan, Horgen (CH); Brandon Morris, Horgen (CH)

(21) Appl. No.: 15/760,914

(22) PCT Filed: Sep. 19, 2016
(86) PCT No.: PCT/US2016/052443

§ 371 Date: Mar. 16, 2018

Related U.S. Application Data
(60) Provisional application No. 62/235,066, filed on Sep. 30, 2015.

(51) Int. Cl. C12Q 1/04; C12Q 1/64; C12Q 1/689; C09K 8/52; E21B 41/00; E21B 49/08

(52) U.S. Cl. CPC C12Q 1/04; C12Q 1/64; C12Q 1/689; E21B 2049/085; E21B 41/00; E21B 49/08; C09K 2208/20; C09K 8/52

(57) ABSTRACT

Methods are provided for detection of souring or the likelihood of souring in an oil and gas process fluid which involves analysis of an aqueous sample obtained from an oil and gas process fluid for the presence of bacteria of at least two different bacterial genera, wherein the genera are selected from Pelobacter, Flexistipes, Marinobacterium, Geoalkalibacter, and Halanaerobium. The presence of bacteria from two or more said genera indicates the likelihood of increased sulfide production in the fluid. Other related methods and reagent compositions for performing the methods are provided.

### BACKGROUND OF THE INVENTION

Currently, the production of oil and gas is becoming increasingly more difficult as secondary recovery techniques are exploited to displace the oil in the reservoirs and maintain reservoir pressure. Many oilfield operations occur with a fluid (generally referred to as “wellbore fluids”) that circulate through or are otherwise introduced into the borehole. These fluids may include drilling fluids that are injected and circulated during the drilling of the well, well stimulation fluids, completion fluids that may be circulated during or after drilling during various completion operations, and fracturing fluids which may be used after drilling in order to stimulate the well to increase production from a hydrocarbon reservoir. The fluids are generally water, often sea water or brine, and can also contain high viscosity fluid additives or friction reducers. As oilfields become more mature, the water production from oil producing wells increases.

The different water streams must be separated from the oil, using topside facilities (separators, deaeretors, pipes, etc.), which often represent a considerable value to a company. The oil which is separated from the water for further refinement is another source of expense. With the introduction of water, (either from the formation or introduced via seawater), microbial contamination is also a problem. Microbial contaminants, particularly bacteria and archaea, can grow and proliferate on the surface or downhole of the well and on drilling and pipeline surfaces.

Most of these oil and gas environments, such as wells, tanks, pipelines and separators are anoxic, elevated in temperature and highly saline. Nitrate addition may take place in parts of the system; and other parts of industrial systems can be aerobic. In facilities or industrial units that serve oil fields (wells, turrets, pipes, separators, etc.), the environment can change dramatically, driven by the amended water chemistry in different elements of the industrial units, the availability of a specific electron acceptor, and the changes of the environmental parameters. Environmental factors, such as high salinities and temperatures, metal pollution, etc. make oil and gas environments ecosystems that encourage microbial growth.

One of the most well-known problems due to microbial contamination includes the formation of H₂S via sulfate reduction (souring). The formation of H₂S via sulfate reduction is often initiated via the introduction of sulfate via seawater injection (the average sulfate concentration in seawater is 2.72 g/kg). Other problems include the degradation of functional chemicals such as corrosion inhibitors, oxygen scavengers, and demulsifiers and biofilm formation that may enhance corrosion and/or hinder proper functioning of equipment (e.g., clogging of filters and valves, decrease heat transfer, etc.).

The biogenic production of H₂S is especially problematic for oil and gas wells because H₂S is highly toxic to humans. The biogenic production of H₂S presents a substantial risk for people working in the industry. Souring also causes degradation of the oil leading to needed desulfurization of oil presenting a significant cost to the oil refinement. Hydrogen sulfide also reacts with metal surfaces causing corrosion, which affects asset integrity storage tanks, pipelines, valves, etc. This accelerated local corrosion of metal surfaces which is caused by the metabolic activity of microorganisms often takes place under a biofilm or other type deposit and puts the physical assets at risk of failure much sooner than their expected and budgeted lifetimes. This has a significant impact on the cost management of above ground facilities. Additionally, increased H₂S levels cause quality degradation of the hydrocarbons as it must be removed again at a later stage (desulfurization). This also has a cost impact on the hydrocarbons eroding the already small margins on hydrocarbons. The production of H₂S and scale deposits in the reservoir as a consequence can cause conformance issues of the reservoir itself (e.g. hindrance of phase flow gas, oil, water, reservoir or the subsurface strata where the hydrocarbons are produced). Souring remains difficult to assess in terms of severity and is even more difficult to detect before it becomes problematic.

Energy and associated service companies often revert to microbial control programs in order to reduce the amounts of microorganisms that grow in the different water phases and exert these activities. Biocides and antimicrobials may be used to inhibit and/or remove microbial growth in the water. The presence of biocides in the water which must be disposed of or recycled back into seawater itself poses an additional problem for the industry. However, it remains difficult to assess the severity of the problem presented by the presence of microorganisms thriving in oil (gas)-water systems as most of these reservoirs and industrial systems cannot be “opened” and are designed to be ‘closed’ systems with limited sample points.

Because many organisms are unculturable, typical microbiology analyses in the petroleum industry include cell density checks determining how many cells are present in the wells, e.g. cells/ml, and ecological studies. While biocide compositions are available that provide adequate biocidal activity in downhole operations, the testing of the oilfield fluids will conventionally take weeks to provide information regarding the efficacy of the biocide. The efficacy of the biocide (or any treatment process) may be affected by the biocide composition, the dosing amount of the biocide, and outside factors such as, but not limited to, chemical and/or sand compositions used in the wellbore composition.

Thus, there is a need for improved technologies which allow the early detection of souring caused by uncontrolled growth of microorganisms in oil and gas facilities. This would enable more targeted use of biocides in the circulating and later recycled water.

### BRIEF DESCRIPTION OF THE DRAWINGS

FIG. 1 is Table 1 that shows the population percentages of relevant genera & species as determined by sequenced DNA in 20 sour shale gas wells.

FIG. 2 is Table 2 which is a summary of Table 1 of the bacterial genera detected in the 20 sour wells, based on different cutoff levels (% of population). A hit in the environment is determined as genera present above a certain percentage which is deemed as relevant.

FIG. 3 is Table 3 that shows the population percentages of relevant genera and species as determined by sequenced DNA in 25 non-sour wells where non-sour wells are defined as those in which no hydrogen sulfide was detected.

FIG. 4 is Table 4 which provides a summary of Table 3 of the genera detected in the 25 non-sour wells, based on different cutoff levels (%).

FIG. 5 is Table 5 which is a summary comparing the indicator genera of non-sour versus sour wells at a 0.5% population cutoff. In this table, the most profound differences can be observed between the presence or absence of these indicator genera in sour versus non sour wells.

FIG. 6A is Table 6A which is a summary comparing the indicator genera of sour versus non-sour wells at the following population cutoffs: No cutoff, 0.01% cutoff, 0.05% cutoff and 0.07% cutoff.

FIG. 6B is Table 6B which is a summary comparing the indicator genera of sour versus non-sour wells at the following population cutoffs: 0.08% cutoff, 0.1% cutoff, 0.5% cutoff and 0.8% cutoff.

FIG. 6C is Table 6C which is a summary comparing the indicator genera of sour versus non-sour wells at the following population cutoffs: 0.9% cutoff, 1% cutoff, and 5% cutoff.

FIG. 7 is Table 7 which shows the side by side comparison of the counts and weighted average of bacterial genera present in sour and non-sour shale gas wells. Highlighted are the organisms that are potential indicator organisms.

FIG. 8 is a chart showing the cut-offs between false positives and false negatives in the non-sour wells (▪) vs. sour wells (▴).

### SUMMARY OF THE INVENTION

The methods described herein permit the operator to detect genetic signatures of souring-causing species before any of the earlier listed issues occur so that preemptive or proactive treatment programs can be started in order to protect hydrocarbons from being reduced in quality, prevent biogenic damage to the reservoir (e.g. caused by biofilms and scale formation) and collection/processing assets from being chemically corroded by H₂S which is biogenically produced by microorganisms. This method and composition relies upon the detection of “indicator organisms” (i.e., bacterial genera and/or species thereof), which when present can be used as a way to predict the potential for souring to enable preventive actions.

In one aspect, a method for the early detection of souring or the likelihood of souring in an oil and gas process fluid comprises preparing an aqueous sample obtained from an oil and gas process fluid comprising bacteria from multiple genera; and analyzing the sample for the presence of bacteria of at least two different genera, wherein the genera are selected from Pelobacter, Flexistipes, Marinobacterium, Geoalkalibacter, and Halanaerobium. The presence of bacteria from two or more said genera indicates the likelihood of increased sulfide production in the fluid.

In another aspect, a method of monitoring the potential for souring in a shale gas well comprises contacting a sample of said well with a reagent composition that can detect the presence of bacteria of at least two different genera, wherein the genera are selected from Pelobacter, Flexistipes, Marinobacterium, Geoalkalibacter, and Halanaerobium; and detecting or measuring a significant concentration of bacteria from at least two of said genera, wherein the presence of bacteria from two or more said genera indicates the likelihood of souring of said wells.

In a further aspect, a method of treating or preventing souring in an oil and gas process fluid comprises preparing an aqueous sample obtained from an oil and gas process fluid comprising bacteria from multiple genera; analyzing the sample for the presence of bacteria of at least two different genera, wherein the genera are selected from Pelobacter, Flexistipes, Marinobacterium, Geoalkalibacter, and Halanaerobium; wherein the presence of bacteria from two or more said genera indicates the occurrence of increasing sulfide production in the fluid; and contacting the well with a broad-spectrum antibiotic or irradiation in an amount sufficient to reduce the growth of said bacteria.

In yet a further aspect, a method for detecting souring or the likelihood of souring in an oil and gas process fluid comprises preparing an aqueous sample obtained from an oil and gas process fluid comprising bacteria from multiple genera; and analyzing the sample for the presence of bacteria of the genus Pelobacter or the genus Flexistipes. The presence of bacteria of either genus is predictive of increased or increasing sulfide production in the fluid.

In yet another aspect, a method of treating or preventing souring in an oil and gas process fluid comprises preparing an aqueous sample obtained from an oil and gas process fluid comprising bacteria from multiple genera; analyzing the sample for the presence of bacteria of the genus Flexistipes or the genus Pelobacter, wherein the presence of bacteria of the either genus is predictive of increased or increasing sulfide production in the fluid and contacting the water in the well with one or more broad-spectrum biocides, bacteriocins or metabolic inhibitors in an amount sufficient to reduce the growth of said bacteria.

Other aspects and advantages of these methods and compositions are described further in the following detailed description.

### DETAILED DESCRIPTION OF THE INVENTION

### Definitions and Components of the Methods and Compositions

Technical and scientific terms used herein have the same meaning as commonly understood by one of ordinary skill in the art to which this invention belongs and by reference to published texts, which provide one skilled in the art with a general guide to many of the terms used in the present application. Any definitions are provided for clarity only and are not intended to limit the claimed invention.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited.

As used herein, the term “souring” means the presence of detectable sulfide, such as H₂S, in a sample from a well or apparatus associated with a well.

As used herein, the term ‘well’ refers to a hydrocarbon (oil and/or gas) producing well, including shale gas wells.

By the term “wellbore fluid” or “oil and gas process fluid” as used herein refers to all fluids containing water, including sea water or brine, used in the oil and gas industry and associated service industries and collected from the wells, or from the physical apparatus associated with oil and gas production as discussed above. These fluids can contain a wide variety of microorganisms from a wide variety of bacterial genera, in various concentrations.

As used herein, the term “pipeline” includes any line in which fluid is moved, including any onshore or offshore flow system, such as mainline systems, risers, flowlines used to transport untreated fluid between a wellhead and a processing facility, and flow lines used to transport treated fluids.

As used herein, the term “sample” refers to a liquid and/or a solid component from an oil and gas process fluid or well bore fluid. If the component is a solid, it may be mixed with water for evaluation. The sample may be obtained from any known source of water used in the oil and gas processes or on or in apparatus used in these processes. The sample may be collected prior to the wellbore fluid entering the wellbore or after the wellbore fluid has been downhole and re-circulated to the surface. Alternatively, the sample may be of water intended for recycling after use or fluid removed from washing or treating the equipment used in these processes. In another embodiment, the sample may be collected after the wellbore fluid has been treated to reduce contaminants.

By “target” is meant a nucleic acid sequence that is found in one or more bacteria from a common genus. In one embodiment the target sequence of bacteria from a single genus is the 16S ribosomal RNA (rRNA) gene sequence. In another embodiment, the target sequence is a single-stranded ribonucleic acid sequence, or a single strand of a deoxyribonucleic acid sequence from one or more bacterial species from one or more of the bacterial genera which compose the indicator microorganisms. Included in this definition are RNA, mRNA, microRNA, a single strand of DNA, cDNA, small-interfering RNA (siRNA), short-hairpin RNA (shRNA), peptide nucleic acid (PNA), transfer RNA (tRNA), ribosomal RNA (rRNA) and DNA from a 16S rRNA gene. In one embodiment, the target sequence is conserved within bacteria from a single genus. In one embodiment, the target is variable within bacteria from a single genus. In another embodiment, the target sequence is a fragment of nucleic acid sequence of a gene. More specifically, the target sequences may be nucleic acid regions of the 16S rRNA gene, or a homolog or naturally occurring ortholog of same that provides a suitable “target” for detection by a nucleic-acid based detection tool, such as qPCR primers, PCR primers or hybridization probes.

As used herein, a target sequence is characteristic of a species of bacteria in one genus selected from Pelobacter, Flexistipes, Marinobacterium, Geoalkalibacter, and Halanaerobium. Various target sequences are from these genera are publically available from databases. By “Pelobacter” is meant any species of the genus Pelobacter. By “Flexistipes” is meant any species of the genus Flexistipes. By “Marinobacterium” is meant any species of the genus Marinobacterium. By “Geoalkalibacter” is meant any species of the genus Geoalkalibacter. By “Halanaerobium” is meant any species of the genus Halanaerobium.

By “microbial control treatment” as used herein means chemicals and biologicals which are used to inhibit or reduce bacterial growth, including conventional biocides, bacteriocins, bacteriophages, and metabolic inhibitors, such as nitrate or nitrite. In addition to biocides, oxygen scavengers are used to remove oxygen from wellbore fluids or oil and gas process fluids. Microbial control treatments must be coordinated with oxygen scavengers to ensure the overall treatments are complimentary. In some embodiments, the microbial control treatment is selected to have minimal or no interaction with any of the components in the wellbore fluid. In other embodiments, the biocide is selected to have interaction with the components in the wellbore fluid. In some embodiments, wellbore fluids may employ either glutaraldehyde or tetrakis-hydroxymethyly-phosphonium sulfate (THPS) to control bacterial contamination. Other examples of chemical biocides include quaternary ammonium biocides, formaldehyde donors, isothiazolone, DBNPA, as well as oxidizing biocides such as sodium hypochlorite, chlorine dioxide, hydrogen peroxide, ozone, and bromine biocides. In some embodiments, wellbore fluids may employ alternative microbial control treatments such as bacteriocins, bacteriophages and/or metabolic inhibitors. In other embodiments, one skilled in the art may determine the type of microbial control treatment to treat the wellbore fluid or oil and gas process fluid based upon the type and amount of the microbial population in the wellbore fluid.

By “alternative microbial control process” as used herein is meant a treatment process other than chemical and biological treatments to produce a treated wellbore fluid. Such treatment processes include equipment-based technologies such as ultra violet radiation (UV); ultra-filtration; thermal; ionizing radiation; and non-ionizing radiation.⁹

The term “reagent” or “device” as used herein can refer to a single nucleic acid sequence, such as a primer, one or multiple sets of forward and reverse primers, labeled primers, primers immobilized on a physical substrate or in an array or microarray, or devices incorporating any of the above for use in detecting contamination of bacteria from one or more of the genera Pelobacter, Flexistipes, Marinobacterium, Geoalkalibacter, and Halanaerobium in wellbore fluids.

The term “primer” as used herein is intended to mean oligonucleotide sequences that can bind to and amplify the target sequence in the performance of PCR or qPCR. In one embodiment, a primer set comprises a forward primer that binds to the coding strand of the target in the 5′ to 3′ direction. In another embodiment, a primer set comprises a reverse primer that binds to the complement of the target sequence in the 3′ to 5′ direction. As used herein the primers may be about 15 to about 50 nucleotides in length, including any intermediate length in-between, including at least 15, 16, 17, 18, 19, 20, 21, 22, 23, 24, 25, 26, 27, 28, 29, 30, 31, 32, 33, 34, 35, 36, 37, 39, 40, 41, 42, 43, 44, 45, 46, 47, 48, 49 or 50 nucleotides in length. Such primers may be employed in solution, e.g., in buffer, or immobilized on a substrate or microarray. In other embodiment, the primers may be labeled with detectable, e.g., fluorescent, labels, e.g., according to the TaqMan@ methodology so that the target-specific oligonucleotides produce a fluorescent signal only when the target DNA is amplified during qPCR, or using fluorescent or other labels such as SYBR® Green I dye.

The term “microarray” refers to an ordered arrangement of hybridizable array elements. In one embodiment, a microarray comprises polynucleotide probes that hybridize to the specified target sequence, on a substrate. In another embodiment, a microarray comprises multiple primers (reverse and forward), optionally immobilized on a substrate.

The term “PCR array” refers to microfluidics card or multiwell plate containing an ordered arrangement of gene-specific forward and reverse primers and fluorescence-labeled probe in each micro-well. This array is used with the sample from the water supply, or a product derived therefrom (e.g., cDNA or cRNA derived from RNA) in real-time PCR reactions which are monitored using fluorescent dye, for example SYBR Green or TaqMan® technology using a reporter fluorescent dye. Each set of primers is designed to amplify the target sequence of interest, such as those described herein. Such primers can readily be designed by one of skill in the art using known techniques, given the instructions on the primer identities described in this specification. Primers may also be prepared or available commercially, for example from Invitrogen: http://bioinfo.invitrogen.com/genome-database/browse/gene-expression/keyword/Taqman %20primers?ICID=uc-gex-Taqman.

The term “real-time quantitative PCR”, or “qPCR” refers to a technique combining PCR amplification and detection into a single step. With qPCR, fluorescent dyes are used to label PCR products during thermal cycling. Real-time PCR instruments measure the accumulation of fluorescent signal during the exponential phase of the reaction for fast, precise quantification of PCR products and objective data analysis. qPCR reaction products are fluorescently labeled using one of two main strategies: Reactions are run in real-time PCR instruments with thermal cycling and fluorescence detection capabilities. By using highly efficient PCR primers and optimal conditions for amplification, every target molecule is copied once at each cycle and data are captured throughout the thermal cycling. Since qPCR reactions are set up with a large molar excess of PCR primers and thermostable DNA polymerase, in the early rounds of thermal cycling, target-template is the limiting factor for the reaction thus, fluorescent signal is directly proportional to the amount of target in the input sample.

As used herein, the term “next generation sequencing” refers to technologies such as the Illumina dye sequencing technologies (Illumina, Inc., San Diego, Calif.) and the 454 parallel pyrosequencing technologies (454 Life Sciences, Roche, Basel, Switzerland), to identify bacteria. Generally the target of such sequencing is the 16S rRNA gene of the bacterium, which is a phylogenetic classifier for species determination. Due to its function in the cellular process it is a highly conserved DNA sequence. The 16S rRNA gene sequences from environmental samples are often used to determine which organisms thrive in these environments. The 16S rRNA genes derived from unknown environments, such as oil or gas associated ecosystems, can provide information on to which known organisms these unknown organism types relate. The retrieved sequence is compared to sequences of known described species in the literature (e.g. via the SILVA database). In common terrestrial ecosystems, the 16S rRNA gene sequence cannot be used a classifier for metabolism or other biological activities.

Methods of Early Detection of and/or Predicting, Souring

The methods and compositions described herein are based upon the discovery that the presence of certain bacterial genera in significant abundance in the indigenous bacterial or archaeal community of an oil and gas process fluid is an accurate predictor of pending or on-going souring-related processes. The methods and compositions described herein enable early detection of souring through detection of these specified contaminants and permit treatment programs to be designed and implemented for eliminating or minimizing metabolic activities of the indicator genera in order to prevent or control souring.

The inventors have determined that the environments of soured wells, wells in the early process of souring, and non-soured wells can be discriminated from each other based on distinct differences in the presence of indicator organisms, i.e., certain genera of bacteria and/or the combinations of certain genera of bacteria. Based on geochemical ecosystem parameters and if the wells are suffering from an issue that correlates to the presence and activities of microbes, their environments become more discriminative from each other and results in differences in concentration in wellbore fluids of bacteria from genera that are found in the waters from these environments. The inventors determined that qualitative and/or quantitative detection of bacteria from one or combinations of the genus Flexistipes, genus Geoalkalibacter, genus Halanaerobium, genus Marinobacterium, and genus Pelobacter may be used to detect wells that are souring. Example 1 below details the discovery.

In one embodiment, a method for detecting souring or the likelihood of souring in an oil and gas process fluid comprises preparing an aqueous sample obtained from an oil and gas comprising bacteria from multiple genera; and analyzing the sample for the presence of bacteria of the genus Pelobacter. The presence of bacteria of the Pelobacter genus is predictive of increased or increasing sulfide production in the fluid.

In another embodiment, a method for detecting souring or the likelihood of souring in an oil and gas process fluid comprises preparing an aqueous sample obtained from an oil and gas comprising bacteria from multiple genera; and analyzing the sample for the presence of bacteria of the genus Flexistipes. The presence of bacteria of the Flexistipes genus is predictive of increased or increasing sulfide production in the fluid.

In yet another embodiment, a method for the early detection of souring or the likelihood of souring in an oil and gas process fluid comprises preparing an aqueous sample obtained from an oil and gas process fluid comprising bacteria from multiple genera; and analyzing the sample for the presence of bacteria of at least two different genera, wherein the genera are selected from Pelobacter, Flexistipes, Marinobacterium, Geoalkalibacter, and Halanaerobium. The presence of bacteria from two or more said genera indicates the likelihood of increased sulfide production in the fluid.

In another embodiment, the methods involve detecting bacteria from at least three of these genera. In another embodiment, the methods involve detecting bacteria from at least four of these genera. In still a further embodiment, the method detects bacteria from all five of these identified genera.

Regarding the embodiment in which combinations of certain genera of bacteria form the discriminating feature, the combination can be detecting bacterial from at least two of these genera. In one embodiment, the method involves the identification of bacteria from both Pelobacter and Flexistipes genera. Additional combinations with Pelobacter and/or Flexistipes can include detection of significant concentrations of bacteria from the genus Marinobacterium or the genus Geoalkalibacter or the genus Halanaerobium. Thus in other embodiments, the method involves identification of bacteria are from the genera Pelobacter and Marinobacterium, or Pelobacter and Geoalkalibacter, or Pelobacter and Halanaerobium. In still other aspects of the invention, the characteristic bacteria identified in the tested fluid are from both genera Flexistipes and Marinobacterium, or Flexistipes and Geoalkalibacter, or Flexistipes and Halanaerobium. In yet a further aspect, the method operates by identifying the presence of significant amounts of bacteria from the genera Marinobacterium and Geoalkalibacter, or Marinobacterium and Halanaerobium, or Geoalkalibacter and Halanaerobium.

Still other embodiments of the invention indicate souring when bacteria from the following three genera are detected in significant amounts: Pelobacter, Flexistipes, and Marinobacterium; or Pelobacter, Flexistipes, and Geoalkalibacter; or Pelobacter, Flexistipes, and Halanaerobium; or Pelobacter, Marinobacterium, and Geoalkalibacter; or Pelobacter, Marinobacterium, and Halanaerobium; or Flexistipes, Marinobacterium, and Geoalkalibacter; or Flexistipes, Marinobacterium, and Halanaerobium; or Marinobacterium, Geoalkalibacter, and Halanaerobium.

In yet other embodiments, the method involves the identification of significant amounts of bacteria from four genera, e.g., Pelobacter, Flexistipes, Marinobacterium and Geoalkalibacter. In another embodiment, the method involves the identification of significant amounts of bacteria from the genera Pelobacter, Flexistipes, Marinobacterium and Halanaerobium. In another embodiment, the method involves the identification of significant amounts of bacteria from the genera Flexistipes, Marinobacterium, Geoalkalibacter and Halanaerobium. In another embodiment, the method involves the identification of significant amounts of bacteria from the genera Pelobacter, Flexistipes, Geoalkalibacter and Halanaerobium.

In yet a further embodiment, the method enables the early detection of souring or permits the prediction of the likelihood of the occurrence of souring when bacteria from all five of the genera Pelobacter, Flexistipes, Marinobacterium, Geoalkalibacter and Halanaerobium are detected in the well.

The steps involved in preparing the sample include extraction of total microbial DNA from oil and gas process fluid samples collected from oil and gas wells or from fluids otherwise used in this industry. The analysis method, in one embodiment, involves sequencing the purified DNA and performing sequence analyses to sequence to identify the bacterial genera composition of the microbial community and determine relative abundance of the identified genera. The detection of the single genus of Pelobacter or Flexistipes in the sample and/or the detection of the combination of these indicator genera enables identification of the well or other source of the sample as ‘sour’, not sour or having a likelihood of progressing to soured fluid.

The analyzing step of each of these methods can involve a quantitative or qualitative detection of said bacteria. In certain embodiments, these methods involve identifying the presence of the bacteria of a genus when the amount or concentration or abundance of each genus is more than 0.05% of the total microbial community in the sample. In another embodiment, the amount of each genus in the methods is more than 0.08% of the total. In yet another embodiment, the amount of each genus in the methods is more than 0.1% of the total. In yet another embodiment, the amount of each genus in the methods is more than 0.2% of the total. In yet another embodiment, the amount of each genus in the methods is more than 0.3% of the total. In yet another embodiment, the amount of each genus in the methods is more than 0.4% of the total. In yet another embodiment, the amount of each genus in the methods is more than 0.5% of the total. In yet another embodiment, the amount of each genus in the methods is more than 0.6% of the total. In yet another embodiment, the amount of each genus in the methods is more than 0.7% of the total. In yet another embodiment, the amount of each genus in the methods is more than 0.8% of the total. In yet another embodiment, the amount of each genus in the methods is more than 0.9% of the total. In yet another embodiment, the amount of each genus in the methods is more than 1% of the total microbial community in the sample. This range of from 0.5 to 1% includes all integers between each of the individually recited numbers and percentages. It is also possible that the presence of a single genus or two or more of the indicator genera may be detected in amounts well over 1%, however, such detection is not necessary for the performance of the methods described herein.

As one specific embodiment, the methods described herein include identifying the potential for souring and/or detecting early souring in an oil and gas process fluid sample by measuring a significant concentration of bacteria from the single genus of Pelobacter or Flexistipes, or the combination of two, three, four or all five bacterial genera identified above, wherein bacteria from each genus are present in said sample at a concentration of greater than about 0.08% of total bacteria of any genus in said sample.

As another specific embodiment, the methods described herein include identifying the potential for souring and/or detecting early souring in an oil and gas process fluid sample by measuring a significant concentration of bacteria from the single genus of Pelobacter or Flexistipes, or the combination of two, three, four or all five bacterial genera identified above, wherein bacteria from each genus are present in said sample at a concentration of greater than about 0.1% of total bacteria of any genus in said sample.

Still other embodiments of these methods are understood by the person of skill in the art given the teachings herein.

The methods described herein are for detecting on-going or future reservoir souring by employing genetic signature detection methods suitable for use in the methods and with the compositions described herein to determine the presence and measure the abundance of at least these five target genera of bacteria comprising genus Flexistipes, genus Geoalkalibacter, genus Halanaerobium, genus Marinobacterium, and genus Pelobacter.

These genetic signature detection or analysis techniques employed in these methods include appropriate known detection methods including but not limited to sequencing and sequence analysis to measure the abundance certain target genera of bacteria in water, other fluids and scrapings or deposits from gas or oil wells, including pipelines, storage containers, or other liquid transporting or liquid holding infrastructure. These analysis techniques can include classic nucleic acid analysis as well as the more recently used next generation sequencing techniques described in the examples below. Thus the analysis, in one embodiment, involves performing any type of RNA or DNA analysis to identify a target sequence in the bacteria present in the sample. In another embodiment, the methods employ performing a polymerase chain reaction (PCR) analysis on the sample. In another embodiment, the methods employ performing a real time qualitative polymerase chain reaction (qPCR) analysis. In yet another embodiment of these methods, next generation dye sequencing, e.g., pyrosequencing, analysis is performed. In still other analysis steps, a microarray analysis is performed on the sample to detect the presence (quantitative or qualitative) of the one or combinations of the five genera of bacteria identified herein.

In one embodiment the analyzing step comprises contacting the sample with a reagent composition or test device that enables qualitative or quantitative detection of nucleic acid sequences that are characteristic of Pelobacter or Flexistipes singly, or combinations of two of more of the five identified genera. In another embodiment, the methods involve confirming the presence of bacteria of at least two said genera and subsequently examining said well by detecting or measuring the amount of sulfide production in said process fluids.

In another embodiment, these methods involve an analysis that targets the 16S ribosomal sequences of bacteria of the targeted genera.

In still another embodiment, the method involves comprises annealing a first primer or probe to a common nucleic acid target sequence present in bacteria of a first genus selected from Pelobacter, Flexistipes, Marinobacterium, Geoalkalibacter, and Halanaerobium at a selected annealing temperature. In another embodiment, another step of the method involves annealing an additional primer or probe to a common nucleic acid target sequence present in bacteria of an additional second, third, fourth or fifth genus selected from Pelobacter, Flexistipes, Marinobacterium, Geoalkalibacter, and Halanaerobium at a selected annealing temperature. In such methods the annealing of the first primer or probe and each additional primer or probe occurs simultaneously or sequentially in the sample.

These methods are useful for monitoring the potential or likelihood of souring in a hydrocarbon-associated environment, including an oil-associated environment or a gas associated environment or a shale gas associated environments. Such environments include but are not limited to oil wells, gas wells and all associated equipment and fluids. In one embodiment, such environments can be those in the early stages of souring with no H₂S present in the produced fluids. Early-stage souring in these environments may not be readily identified as soured by other known conventional methods of detecting souring. Thus these methods permit early detection or prediction of souring of, e.g., a well, in the same manner as the methods may be used for confirming already soured or non-soured well samples.

In yet a further aspect, a method of treating or preventing souring in an oil and gas process fluid comprises preparing an aqueous sample obtained from an oil and gas comprising bacteria from multiple genera. The sample is then analyzed for the presence of bacteria of a single genus of Pelobacter or Flexistipes or for combination of two or more different bacterial genera selected from Pelobacter, Flexistipes, Marinobacterium, Geoalkalibacter, and Halanaerobium. The presence of bacteria from the indicative genus or genera combination indicates the occurrence of increasing sulfide production in the sample. The wellbore fluid may then be contacted with one or a combination of biocides, metabolic inhibitors. Alternatively the wellbore fluid may be treated with an alternative method described above, such as ultra violet radiation, ultra-filtration, thermal radiation, ionizing radiation, and non-ionizing radiation, in an amount sufficient to reduce the growth of said bacteria.

After the presence and/or concentration of these indicator bacteria genus or genera are determined, subsequent treatment and testing may be repeated until the microbial population is at an acceptable level so as to reduce the potential for sulfide production. The subsequent treatment may be the same as the initial treatment or may be changed depending on the efficacy of the initial treatment fluid.

An on-site way of providing results of souring activity in the wellbore fluid may provide operators with information for adjusting the biocide composition (or treatment process) or the amount without the delay of shipping sample volumes offsite and would also reflect real time microbial content as compared to a delayed microbial content. With the knowledge of the microbial population activity, formulation of the wellbore fluid can be adjusted and monitored to provide a type of treatment to minimize fouling in the wellbore operations.

These methods also allow for designing and implementing microbial control treatment programs to reduce or prevent detrimental effects of souring.

Reagents

In one embodiment, based upon the type of analysis and the specific method selected as defined above, a reagent composition or a kit for use in the early identification of the condition of souring or the likelihood of souring in a shale gas wells comprises at least one ribonucleic acid sequence or at least one deoxyribonucleic acid sequence useful in identifying the relevant genus. In one embodiment a reagent composition or kit contains a first nucleic acid primer or probe sequence having high specificity for a first nucleotide sequence common to bacteria of a first genus selected from Pelobacter or Flexistipes.

In another embodiment a reagent composition or kit contains a first nucleic acid primer or probe sequence having high specificity for a first nucleotide sequence common to bacteria of a first genus selected from Pelobacter, Flexistipes Marinobacterium, Geoalkalibacter, and Halanaerobium and an additional nucleic acid primer or probe sequence having high specificity for an additional nucleotide sequence present in additional genus selected from Pelobacter, Flexistipes, Marinobacterium, Geoalkalibacter, and Halanaerobium. Second, third, fourth and fifth “additional” sequences can be present in other embodiments to account for combinations of three, four or all five of these indicator genera.

In these reagent compositions or kits comprising multiple reagents, each reagent sequence (primer, probe, etc.) does not cross-react, or minimally cross-reacts, with a bacterium from more than one of the five indicator genera.

In still other embodiments, a reagent composition or kit can contain one or more components selected from a label, a physical substrate, a label component capable of interacting with the label and generating a detectable signal.

In yet another embodiment, a reagent comprises a physical substrate upon which one or more of the nucleotide sequences are immobilized or fixed. Such a substrate can be an array, a microarray, a microchip, a plastic surface, a disk or a glass surface or other known substrate. Association of the sequences on the substrate and methods for accomplishing the association are well known in the art. Alternatively, the primer sequences may be provided in a suitable buffer depending upon the type of method in which the reagent is to be used. In still another embodiment, the reagent includes a primer to which a detectable label or label component is associated. Suitable detectable labels include fluorescent labels such as those employed by the TaqMan® reagents, or other known fluorescent molecules. Suitable labels may be selected from among many known label types by one of skill in the art given the teachings of this specification.

In yet another embodiment, a reagent of this invention can be in the form of a kit containing one or more of the primers, reverse and forward primer sets or labeled primer-probes, suitable labels and labeling methodologies, suitable substrates, and/or label component, e.g., enzymes, capable of interacting with the label associated with a primer and generating a detectable signal. In yet another embodiment, the reagent is configured as a water monitoring device comprising the primers described herein.

In still another embodiment a reagent composition comprises a first nucleic acid primer or probe sequence having high specificity for binding to or associating with a first target nucleotide sequence common to multiple species of a first genus selected from Pelobacter, Flexistipes, Marinobacterium, Geoalkalibacter, and Halanaerobium. In still another embodiment, a reagent composition comprises an additional nucleic acid primer or probe sequence having high specificity for binding to or associating with an additional nucleotide sequence present in multiple species of a second, third, fourth or fifth genus selected from Pelobacter, Flexistipes, Marinobacterium, Geoalkalibacter, and Halanaerobium.

In another embodiment, the reagent composition includes a set of primers comprising a forward nucleotide sequence primer and a reverse nucleotide sequence primer for detecting a bacterial from a single indicator genus or for each indicator genus. The reagent composition, in another embodiment further comprises a substrate upon which one or more of the probes or primers are immobilized or fixed. In still another embodiment, the reagent composition further comprises a detectable label or label component associated with at least one primer or probe.

In some embodiments, the above method may be performed using a test kit that contains the sequences, primers or probes of the selected reagent composition. Other optional components of the kit may include a sterile filter device comprising a filter with a pore size sufficiently small to retain the contaminants on the filter's influent side, means for passing a known volume of sample through the filter (e.g. a syringe), labels for association with the sequences (primers, probes etc.) in which the label that upon interaction with the contaminants will release a detectable moiety, the amount of which can be correlated with the amount of contaminants that have interacted with the primer or probe, and instructions that sets forth steps for performing the desired method.

The above described methods and test kit may be used on-site at oil or gas processing sites.

One of skill in the art may construct a number of suitable reagent compositions for use in the methods described herein given the teachings of this specification.

In order to further understand the above-described device and to demonstrate how the method may be carried out in practice, certain embodiments will now be described with reference to the accompanying drawings above and the examples described below. The following examples are provided for illustration and do not limit the disclosure or scope of the claims and specification.

### Example 1

Using a database which holds 16S rRNA gene sequence distributions from over 500 samples retrieved from various petroleum associated environments, the inventors determined the following.

The data contained in the database was subjected to the following. Paired comparisons were made of microbial communities between shale gas wells which experience souring (H₂S emissions) vs. wells which do not suffer from souring based on reported presence or absence of hydrogen sulfide. The indicative bacterial genera were identified from a representative number of samples by genus based on their 16S rRNA gene sequences, e.g., utilizing next NGS platforms. The results demonstrated it was possible to partition genera according to specific habitats—souring-related genera were detected to be present in a significant concentration in samples from locations where souring was also confirmed by other means. These combinations of genera form the basis of the methods described herein which employ them as biomarker(s) or indicators for the occurrence of souring.

Specifically, the analysis was performed as follows:

Oil field production fluids were sampled from wells and topside facilities into pre-sterilized vessels. The oil and water within the fluids were separated, if needed, and 1 L of water from each sample was filtered through 0.45 μM Pall® filters. Deoxyribonucleic acids (DNA) were extracted from each sample using a MoBio PowerSoil DNA Isolation Kit (MoBio Laboratories, CA-USA) according to the manufacturer's instructions.

After quantification by spectrophotometry, 100 ng of DNA was amplified with barcoded primers using 2.5 units of AmpliTaq (ABI,CA-USA) in a reaction buffer containing 25 mM MgCl2, 1% Triton, 10 mM dNTPs, and 10 mg/ml BSA. PCR was performed on a thermocycler using the following conditions: an initial denaturing at 95° C. for 5 minutes, followed by 20 cycles of 95° C. for 30 seconds, 56° C. for 30 seconds, and 72° C. for 90 seconds. The final reaction was terminated after an 8 minute extension at 72° C. The amplicons from each DNA sample were gel purified using an 0.8% agarose gel and a QIAquick Gel Extraction Kit (Qiagen) according to the vendor's method. The 16S rDNA genes for both bacteria and archaea were targeted using the 515F and 806R primer pair.

For 454 pyrosequencing using the GS FLX technology, library preparation was performed with the 270 bp PCR-fragments, obtained from the initial polymerase chain reaction (PCR) after gel purification, using the GS FLX Titanium Rapid Library Preparation Kit (Roche) according to manufacturer's instructions, in order to prepare single-stranded DNA fragments for further processing. Emulsion-based clonal amplification (emPCR) was used to attach the single-stranded DNA fragments onto DNA capture beads (454™). The DNA capture beads were then pipetted into the 454 Sequencing™ PicoTiterPlate™ and sequenced using manufacturer suggested operating parameters. The resulting flowgrams were assigned nucleotide sequences using the software provided with the instrument. The resulting 16S rDNA sequences, for the mixed microbial communities contained in the samples, were identified and classified by comparing the sequences to the SILVA full-length 16S database maintained at the Max Planck Institute for Marine Microbiology in Bremen, Germany.

### Example 2

Sequence-based microbial diversity profiling as described above of 45 shale gas wells from multiple various locations were completed. Of those wells, 20 were soured and 25 were seemingly free of souring. The sequence analysis results identified a limited number of bacterial genera that were present in ‘souring affected systems’ but absent or close to absent in souring-free systems. The problematic bacterial genera detected and identified are listed below (with details). All of these genera identified in Table 8 were either absent or present in insignificant quantities in the non-soured wells.

TABLE 8

| | Sour wells - 20 in total | | |
|---|---|---|---|
| | | Average | |
| | | abundance | |
| | Presence in | when | |
| | analyzed | present | |
| Genera* | samples | (%) | Known Metabolic activity |
| Halanaerobium | 17 (85%) | 22.58 | Fermenting microorganisms |
| | | | which promote SRB |
| | | | growth, often halophilic |
| Geoalkalibacter | 11 (55%) | 12.24 | Metal reducing species |
| | | | (Fe(III), Mn(IV) |
| Marinobacterium | 13 (65%) | 9.36 | General heterotrophic |
| | | | bacteria, some have |
| | | | capability to utilize |
| | | | atypical substrates |
| Pelobacter | 18 (90%) | 6.48 | Metal reducing species |
| | | | (Fe(III) |
| Flexistipes | 15 (75%) | 5.44 | Fermenting microorganisms |
| Total Genera | 20 (100%) | 41.9 | |

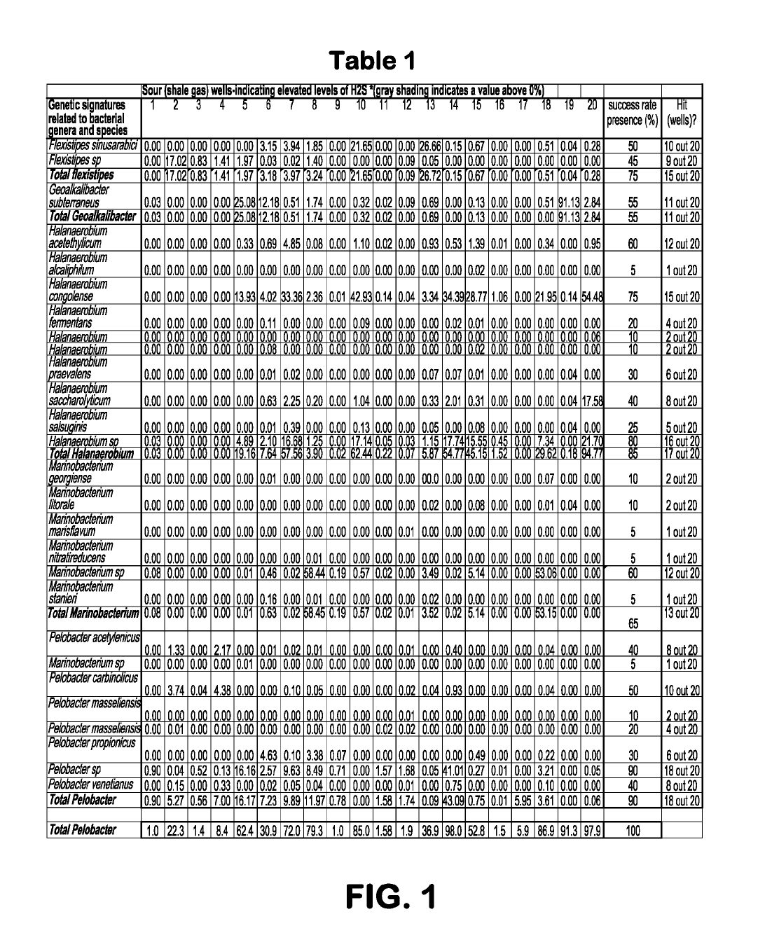

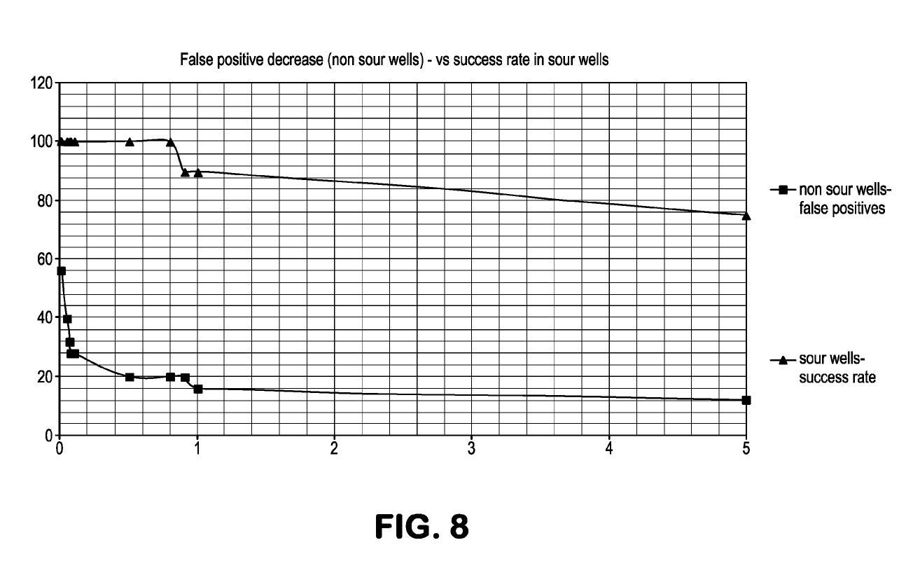

Additional data were analyzed and summaries are presented in FIGS. 1 through 7, i.e., Tables 1 through 7, and FIG. 8, which demonstrate:

(a) When one or more of the genera indicated as problematic is present above 0.5% of the total population, there is a heightened probability that a souring process is taking place or will take place in the future. Based on the twenty sour wells, analysis shows 100% correlation with souring. When we increase the cutoff to 1%, we identify 90% of the soured wells represented in the database and at 5% we identify 75%.

(b) When the sum of the found genera indicated as problematic in total (as part of the total population distribution) is above 1, based on these 20 wells, we identify 100% of the sour wells. As the cutoff of the combined genera population is increased above 1%, we see a similar trend as with the individual genera above.

In order to complete the dataset, we also looked at the non sour wells and the presence of the indicator genera in the wells designated as being non-sour. In about 50% of the non-sour wells, a minute quantity of the genera indicated as souring predictors are present as part of the population distribution. In most cases, the percentages at which these are present are non-relevant. When the same cutoffs are applied, 0.5% for the genera as stand-alone and 1% for the combined genera, 5 out of the 25 non sour wells would still be identified as being sour.

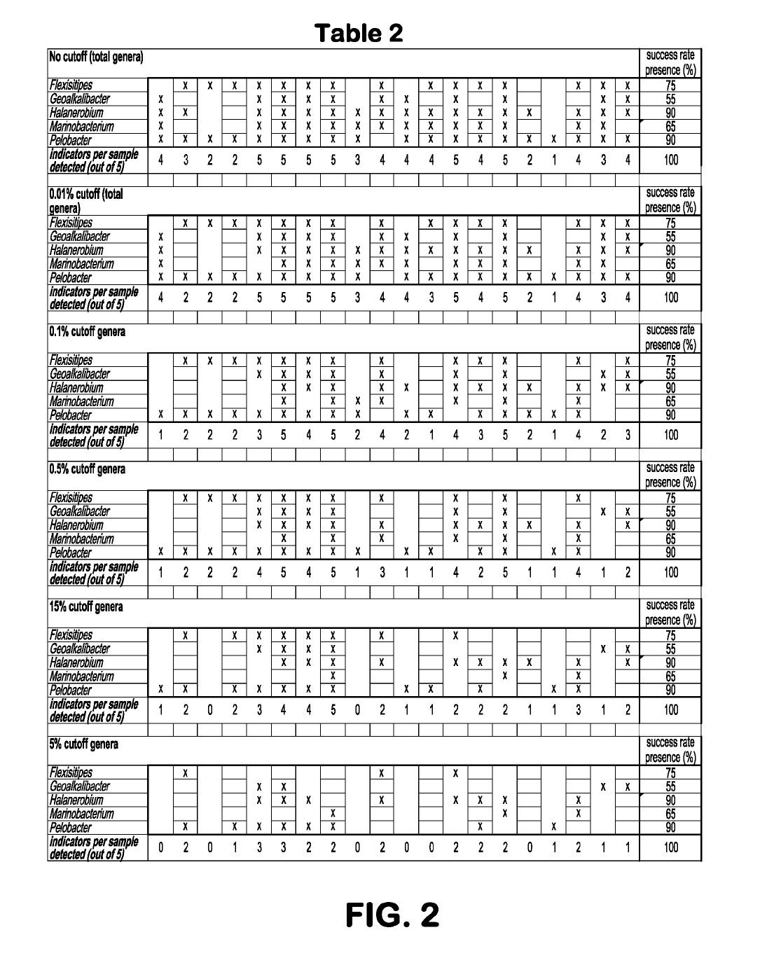

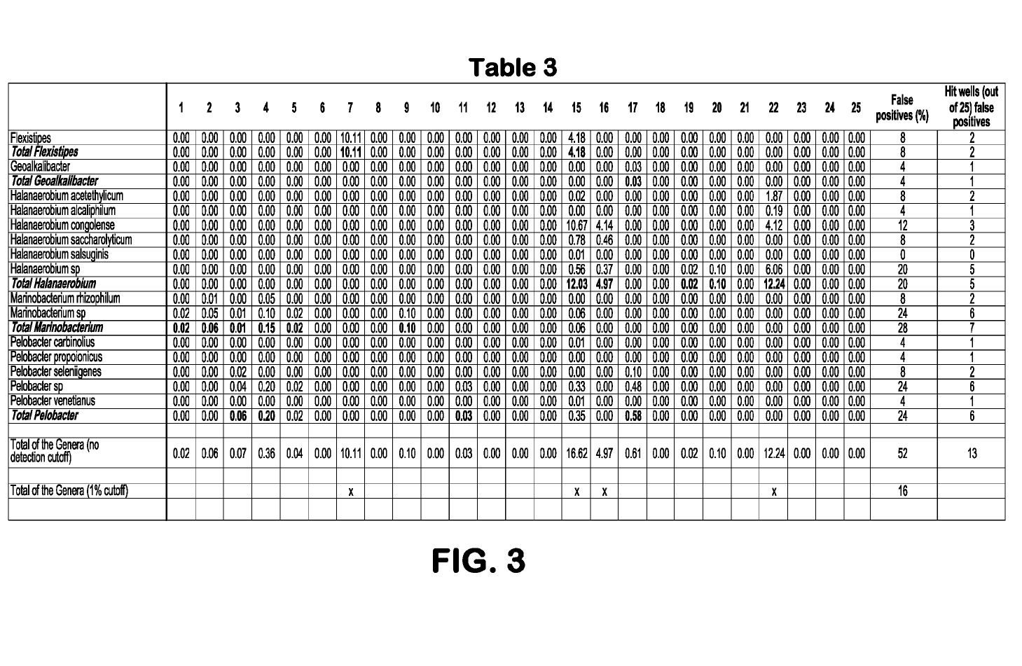

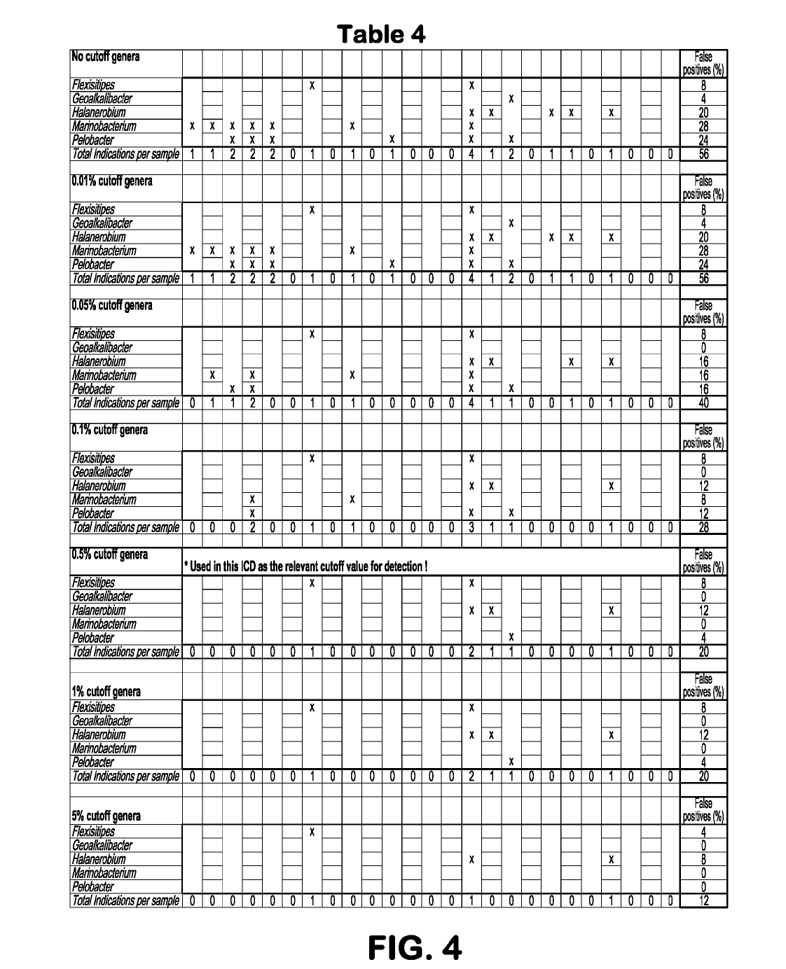

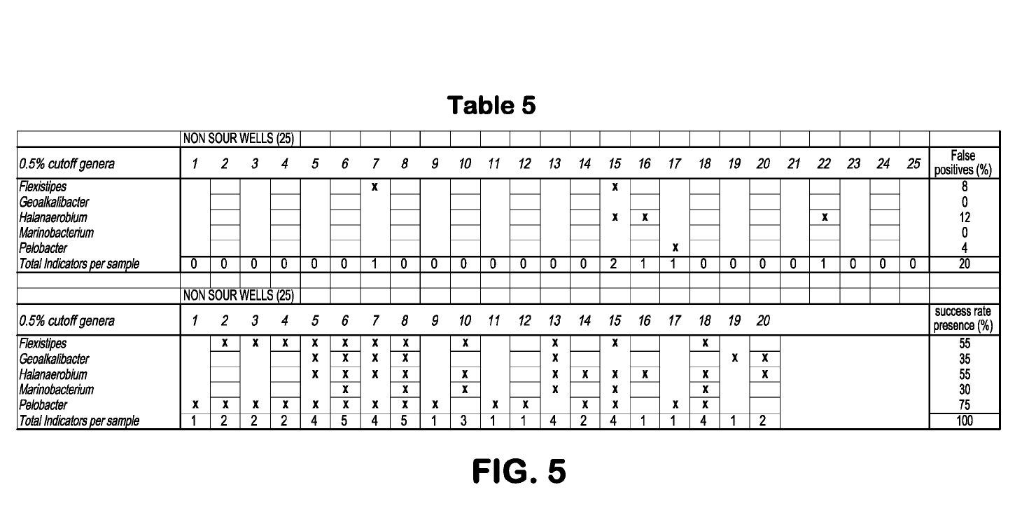

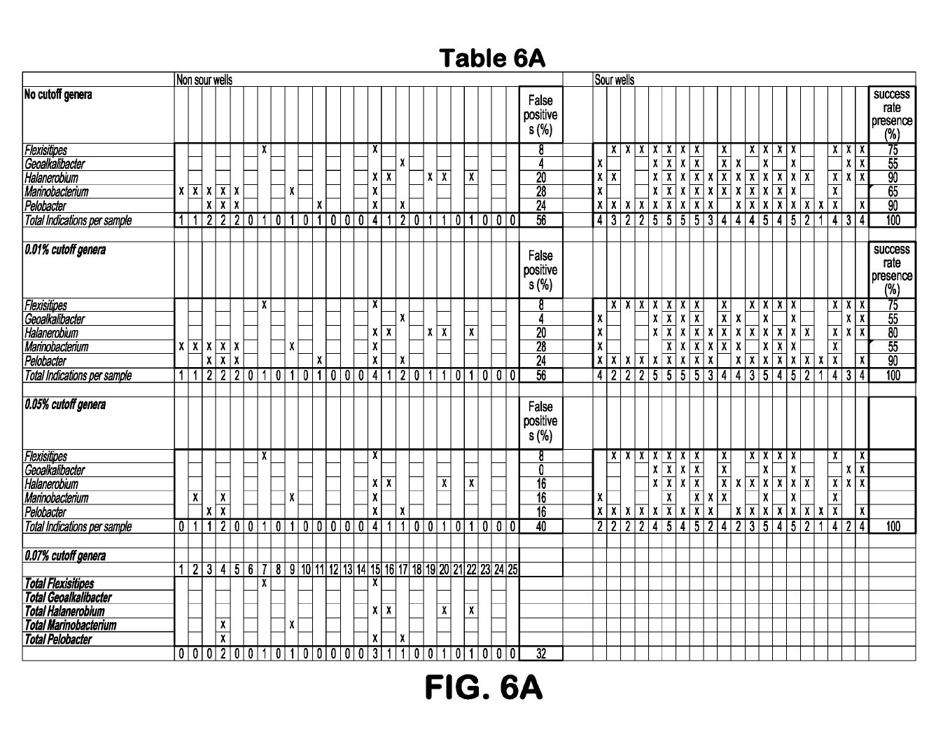

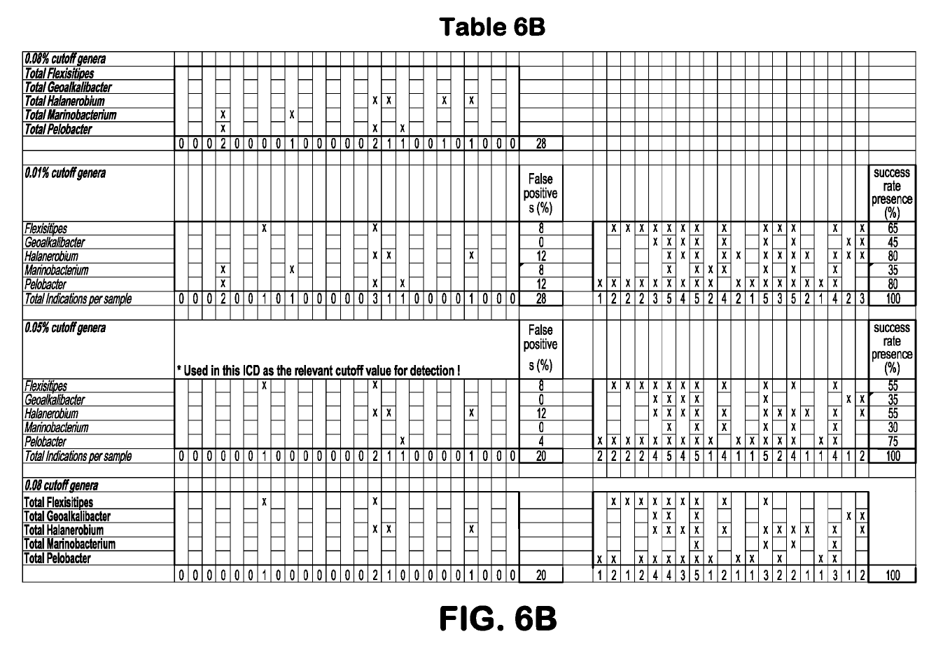

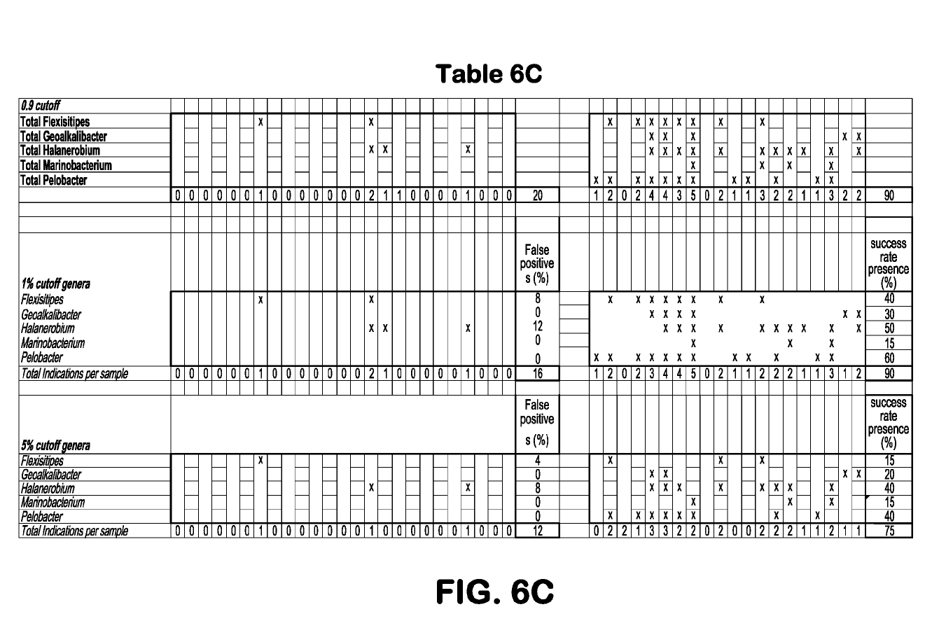

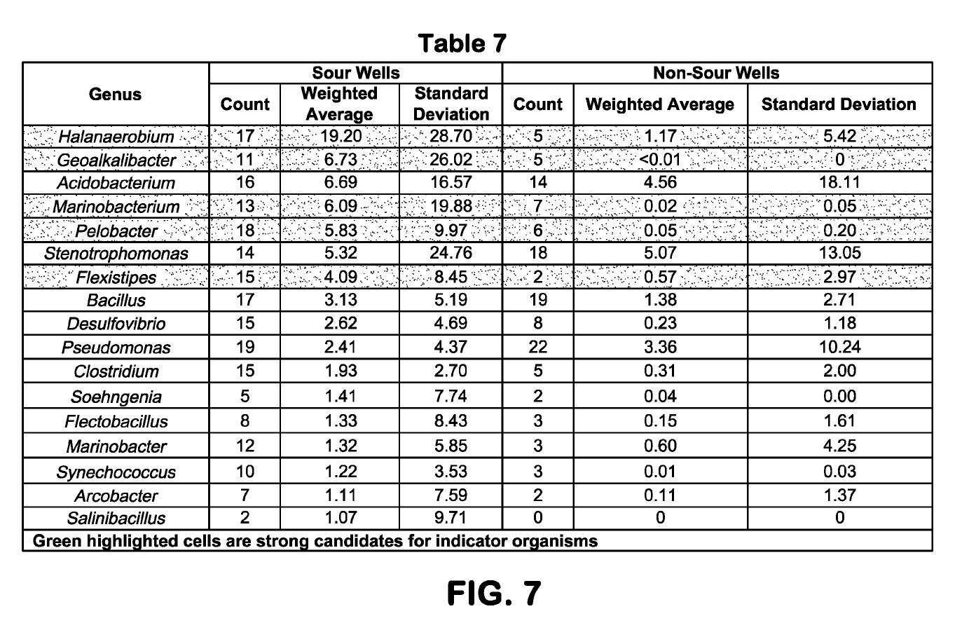

FIGS. 1 and 2 list the data for the sour wells. FIGS. 3 and 4 list all the information from the non sour wells. FIGS. 5 and 6A-6C show the comparative differences in the sour vs. the non-sour wells for the identified population cutoffs. FIG. 7 shows the other bacteria genera preset in the wells, including the non-indicator genera. FIG. 8 shows the cut-offs between false positives and negatives.

Numerous modifications and variations of the embodiments illustrated above are included in this specification and are expected to be obvious to one skilled in the art. Such modifications and alterations to the compositions and processes described herein are believed to be encompassed in the scope of the claims appended hereto. All documents, including patents, patent applications and publications, and non-patent publications listed or referred to below, as well as the attached figures, are incorporated herein by reference in their entireties to the extent they are not inconsistent with the explicit teachings of this specification.

1. A method for the early detection of souring or the likelihood of souring in an oil and gas process fluid comprising:
preparing an aqueous sample obtained from an oil and gas process fluid comprising bacteria from multiple genera; and
analyzing the sample for the presence of bacteria of at least two different genera, wherein the genera are selected from Pelobacter, Flexistipes, Marinobacterium, Geoalkalibacter, and Halanaerobium;
wherein the presence of bacteria from two or more said genera indicates the likelihood of increased sulfide production in the fluid.

2. The method according to claim 1, wherein the analyzing step comprises a quantitative or qualitative detection of said bacteria.

3. The method according to claim 1, further comprising detecting bacteria from at least three said genera.

4. The method according to claim 1, further comprising detecting bacteria from at least four said genera.

5. The method according to claim 1, further comprising detecting bacteria from all five said genera.

6. The method according to claim 1, wherein the analyzing comprises contacting the sample with a reagent composition or test device that enables qualitative or quantitative detection of nucleic acid sequences that are characteristic of at least two of said genera.

7. The method according to claim 1, further comprising measuring a significant concentration of bacteria from said two, three, four or all five bacterial genera, wherein bacteria from each genus are present in said sample at a concentration of greater than about 0.08% of total bacteria of any genus in said sample.

8. The method according to claim 1, further comprising the steps of confirming the presence of bacteria of at least two said genera and subsequently examining said well by detecting or measuring the amount of sulfide production in said process fluids.

9: The method according to claim 1, wherein said analyzing comprises performing a DNA analysis, a polymerase chain reaction (PCR) analysis or a real time qualitative polymerase chain reaction (qPCR) analysis on said sample.

10. The method according to claim 1, wherein said analyzing comprises performing a next Generation pyrosequencing analysis or microarray analysis on said sample.

11. A method of monitoring the potential for souring in a shale gas well comprising:
contacting a sample of said well with a reagent composition that can detect the presence of bacteria of at least two different genera, wherein the genera are selected from Pelobacter, Flexistipes, Marinobacterium, Geoalkalibacter, and Halanaerobium;
detecting or measuring a significant concentration of bacteria from at least two of said genera, wherein the presence of bacteria from two or more said genera indicates the likelihood of souring of said wells.

12. A method of treating or preventing souring in an oil and gas process fluid comprising:
(a) preparing an aqueous sample obtained from an oil and gas process fluid comprising bacteria from multiple genera;
(b) either
i. analyzing the sample for the presence of bacteria of at least two different genera, wherein the genera are selected from Pelobacter, Flexistipes, Marinobacterium, Geoalkalibacter, and Halanaerobium; wherein the presence of bacteria from two or more said genera indicates the occurrence of increasing sulfide production in the fluid; or
ii. analyzing the sample for the presence of bacteria of the genus Pelobacter; wherein the presence of bacteria of the Pelobacter genus is predictive of increased or increasing sulfide production in the fluid; or
iii. analyzing the sample for the presence of bacteria of the genus Flexistipes; wherein the presence of bacteria of the Flexistipes genus is predictive of increased or increasing sulfide production in the fluid; and
(c) contacting the well with a microbial control treatment in an amount sufficient to reduce the growth of said two or more genera, or only of Flexistipes or only of Pelobacter bacteria.

13-14. (canceled)

15. The method according to claim 12
wherein
the well is contacted with a microbial control treatment in an amount sufficient to reduce the growth of said bacteria of the Flexistipes genus.

16. The method according to claim 12
wherein
the well is contacted with a microbial control treatment in an amount sufficient to reduce the growth of said bacteria of the Pelobacter genus.

17. The method according to claim 12, wherein the well is contacted with a microbial control treatment in an amount sufficient to reduce the growth of said two or more genera of bacteria.
